(19) United States
(12) Patent Application Publication
(10) Pub. No.: US 2014/0112445 A1
(43) Pub. Date: Apr. 24, 2014

(54) X-RAY DIAGNOSTIC APPARATUS

(71) Applicants: Toshiba Medical Systems Corporation, Otawara-shi (JP); KABUSHIKI KAISHA TOSHIBA, Minato-ku (JP)

(72) Inventors: Satoshi YAMASHITA, Utsunomiya-shi (JP); Takuya Sakaguchi, Utsunomiya-shi (JP); Ryuji Zaiki, Utsunomiya-shi (JP)

(73) Assignees: Toshiba Medical Systems Corporation, Otawara-shi (JP); KABUSHIKI KAISHA TOSHIBA, Minato-ku (JP)

(21) Appl. No.: 14/144,983

(22) Filed: Dec. 31, 2013

Related U.S. Application Data
(63) Continuation of application No. PCT/JP2013/063142, filed on May 10, 2013.

(30) Foreign Application Priority Data
May 23, 2012 (JP) 2012-117139

(51) Int. Cl. A61B 6/00

(52) U.S. Cl. CPC A61B 6/547
USPC 378/62

(57) ABSTRACT

In an embodiment, an X-ray diagnostic apparatus includes an X-ray irradiator which irradiates a subject with X-rays, an X-ray detector which detects the X-rays emitted from the X-ray irradiator and passing through the subject, a movement mechanism which changes a position of the X-ray detector relative to the subject, a display unit which displays an X-ray image based on the X-rays detected by the X-ray detector, a region setting unit which sets a region of interest in the X-ray image displayed in a display screen of the display unit, and a movement control unit which controls the movement mechanism in such a way that the region of interest set by the region setting unit is kept in the display screen.

### CROSS-REFERENCE TO THE RELATED APPLICATION

This application is based on and claims the benefit of priority from International Application No. PCT/JP2013/063142, filed on May 10, 2013 and Japanese Patent Application No. 2012-117139, filed on May 23, 2012; the entire contents of which are incorporated herein by reference.

### FIELD

Embodiments described herein relate generally to an X-ray diagnostic apparatus.

### BACKGROUND

An X-ray diagnostic apparatus is an apparatus configured to acquire X-ray images of a subject such as a patient by irradiating the subject with X-rays from an X-ray irradiator, and detecting the X-rays that pass through the subject by use of an X-ray detector. As the X-ray diagnostic apparatus of this type, for example, there has been developed an X-ray diagnostic apparatus provided with a C-arm or the like which holds the X-ray irradiator and the X-ray detector in a mutually opposed manner, and configured to capture an X-ray image of a region of attention of a subject on a tabletop by moving the X-ray irradiator and the X-ray detector to positions to capture the image, and to display the image on a monitor.

The X-ray diagnostic apparatus has a normal image capturing mode, and an X-ray irradiation mode called a fluoroscopic mode designed to consecutively display X-ray images of the subject by consecutively emitting small amounts of X-rays from the X-ray irradiator. The fluoroscopic mode is used to find a target region or to observe moving images of a region in motion. X-ray images captured in the fluoroscopic mode are generally referred to as fluoroscopic images.

In addition, a proposal has been made on a technique (for example, a spot imaging method) of: setting a region of interest (ROI) in a fluoroscopic image stored in advance; acquiring fluoroscopic images (moving images) of the region of interest by irradiating only the set region of interest with X-rays; and displaying the moving images superposed on the fluoroscopic image (still image) stored in advance.

There is a procedure of performing a cardiovascular treatment and a surgical operation simultaneously (for example, a hybrid operation) by using such an X-ray diagnostic apparatus. In this procedure, for example, a surgeon in charge of a surgical operation performs a thoracotomy, and an internist in charge of a cardiovascular treatment simultaneously performs a catheter treatment. The catheter treatment such as ablation using a catheter employs the above-mentioned technique of displaying the moving images while superposing them on the still image.

During the above-described procedure, however, a hand of the surgeon and the X-ray detector (or the X-ray irradiator) may come close to each other when the surgeon is performing the operation since the surgeon and the internist perform their operations concurrently. In this case, the X-ray detector (or the X-ray irradiator) is an obstacle to the operation by the surgeon. Nonetheless, if the surgeon moves the X-ray detector (or the X-ray irradiator) at his/her discretion, then the internist may be unable to visually check the region of interest as the X-ray images under observation by the internist may deviate from the relevant region.

### BRIEF DESCRIPTION OF THE DRAWINGS

FIG. 1 is a diagram showing a schematic configuration of an X-ray diagnostic apparatus of an embodiment.

FIG. 2 is an explanatory diagram for explaining a moving direction of an X-ray detector included in the X-ray diagnostic apparatus shown in FIG. 1.

FIG. 3 is a flowchart showing a flow of a first movement process which the X-ray diagnostic apparatus shown in FIG. 1 performs on the X-ray detector.

FIG. 4 is an explanatory diagram for explaining the flow of the first movement process shown in FIG. 3.

FIG. 5 is an explanatory diagram for explaining moving directions of a holding unit and the X-ray detector included in the X-ray diagnostic apparatus shown in FIG. 1.

FIG. 6 is a flowchart showing a flow of a second movement process which the X-ray diagnostic apparatus shown in FIG. 1 performs on the X-ray detector.

FIG. 7 is an explanatory diagram for explaining the flow of the second movement process shown in FIG. 6.

### DETAILED DESCRIPTION

In an embodiment, an X-ray diagnostic apparatus includes: an X-ray irradiator configured to irradiate a subject with X-rays; an X-ray detector configured to detect X-rays emitted from the X-ray irradiator and passing through the subject; a movement mechanism unit configured to change positions of the X-ray detector and the X-ray irradiator relative to the subject; a display unit configured to display an X-ray image on the basis of the X-rays detected by the X-ray detector; a region setting unit configured to set a region of interest in the X-ray image displayed on a display screen of the display unit; and a movement control unit configured to control the movement mechanism unit so as to keep the region of interest within the display screen.

Descriptions will be provided for the embodiment by referring to the drawings.

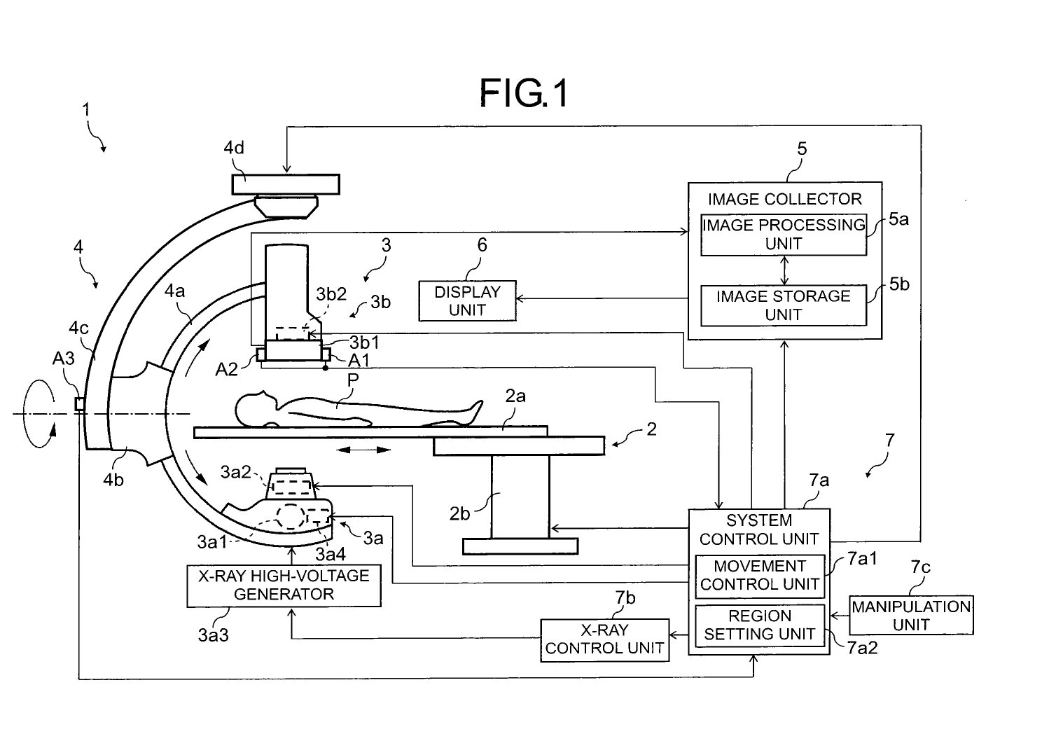

As shown in FIG. 1, the X-ray diagnostic apparatus 1 of the embodiment includes: a bed 2 on which to place a subject P such as a patient; an image capturing device 3 configured to capture images of the subject P on the bed 2; a holding unit 4 configured to hold the image capturing device 3; an image collector 5 configured to collect the X-ray images; a display unit 6 configured to display various images inclusive of the X-ray images; and a control device 7 configured to control the above-mentioned components.

The bed 2 includes: a rectangular tabletop 2a on which to place the subject P; and a tabletop driving unit 2b configured to support and move the tabletop 2a in a horizontal direction and in a vertical direction. The tabletop driving unit 2b includes: a movement mechanism configured to move the tabletop 2a; and a driving source configured to supply driving force for moving the tabletop 2a. The bed 2 moves the subject P on the tabletop 2a to a desired position by causing the tabletop driving unit 2b to move the tabletop 2a to a desired height, and subsequently in the horizontal direction.

The image capturing device 3 includes an X-ray irradiation unit 3a configured to irradiate the subject P on the tabletop 2a with X-rays; and an X-ray detection unit 3b configured to detect the X-rays passing through the subject P. The image capturing device 3 is provided to be movable around the tabletop 2a. Thus, the image capturing device 3 captures X-ray images of the subject P on the tabletop 2a from an image-capturing position. Examples of the X-ray images include fluoroscopic images of a region of attention that covers the heart and the like.

The X-ray irradiation unit 3a includes: an X-ray irradiator 3a1 configured to emit X-rays, such as an X-ray tube; an X-ray diaphragm 3a2 configured to adjust the irradiation field (irradiation range) of the X-rays by partially stopping the X-rays emitted from the X-ray irradiator 3a1; an X-ray high-voltage generator 3a3 configured to generate a high voltage to be supplied to the X-ray irradiator 3a1; and the like. The X-ray irradiation unit 3a causes the X-ray high-voltage generator 3a3 to supply the high voltage to the X-ray irradiator 3a1; causes the X-ray irradiator 3a1 to emit the X-rays; causes the X-ray diaphragm 3a2 to partially stop the X-rays; and eventually irradiates the subject P on the tabletop 2a with the X-rays.

In this respect, various types of X-ray diaphragms may be used as the X-ray diaphragm 3a2. For example, an X-ray diaphragm may be used in which: four X-ray blocking members made of lead or the like are arranged in parallel crosses; and the position and size of a window formed by being surrounded by the X-ray blocking members are changed as needed by moving the X-ray blocking members in such a manner as to be brought closer to or be away from each other. The window portion constitutes a passage area where the X-rays pass through, while the X-ray blocking members located around the window constitute a blocking area for absorbing and blocking the X-rays.

In addition, the X-ray irradiation unit 3a includes a movement mechanism (third movement mechanism) 3a4 configured to move the X-ray irradiator 3a1 and the X-ray diaphragm 3a2 on a plane (for example, a horizontal plane). The movement mechanism 3a4 is a mechanism configured to move the X-ray irradiator 3a1 and the X-ray diaphragm 3a2 in the horizontal direction. The movement mechanism 3a4 is electrically connected to the control device 7, and the drive of the movement mechanism 3a4 is controlled by the control device 7. As the movement mechanism 3a4, for example, a feed screw-type movement mechanism using a servomotor as its driving source, a linear motor-type movement mechanism using a linear motor as its driving source, or the like may be used. Incidentally, the above-described movement mechanism 3a4 functions as the movement mechanism unit configured to change the position of the X-ray irradiator 3a1 relative to the subject P.

The X-ray detection unit 3b includes: an X-ray detector 3b1 configured to detect the X-rays, such as FPD (X-ray flat panel detector); a movement mechanism (first movement mechanism) 3b2 configured to move the X-ray detector 3b1 on a plane (for example, a horizontal plane); and the like.

The X-ray detector 3b1 is provided to the holding unit 4 while facing the X-ray irradiator 3a1, and is formed movably on a plane parallel to the tabletop 2a. The X-ray detector 3b1 is electrically connected to the image collector 5, and transmits detected X-ray doses, namely X-ray image signals, to the image collector 5.

Multiple movement manipulation detectors A1, A2 configured to detect movement manipulations by operators such as a surgeon and an assistant are provided on lateral sides of the X-ray detector 3b1. The movement manipulation detectors A1, A2 are electrically connected to the control device 7, and detect the movement manipulations by the operators, and transmit detection signals to the control device 7. As the movement manipulation detectors A1, A2, for example, a contact manipulation switch like a touch sensor, or a non-contact manipulation switch may be used.

The movement mechanism 3b2 is a mechanism configured to move the X-ray detector 3b1 in the horizontal direction. The movement mechanism 3b2 is electrically connected to the control device 7, and the drive of the movement mechanism 3b2 is controlled by the control device 7. As the movement mechanism 3b2, for example, a feed screw-type movement mechanism using a servomotor as its driving source, a linear motor-type movement mechanism using a linear motor as its driving source, or the like may be used. Incidentally, the above-described movement mechanism 3b2 functions as the movement mechanism unit configured to change the position of the X-ray detector 3b1 relative to the subject P.

The holding unit 4 includes: a holding arm 4a configured to hold the X-ray irradiator 3a1 and the X-ray detection unit 3b while arranging them face-to-face; an arm supporting unit 4b configured to support the holding arm 4a slidably; a holding member 4c movably and turnably provided to a ceiling surface and configured to turnably hold the arm supporting unit 4b; and a movement mechanism (second movement mechanism) 4d configured to move the holding member 4c in the horizontal direction.

The holding arm 4a is a C-arm shaped like the letter C, for example. The holding arm 4a is provided to the arm supporting unit 4b in such a manner as to be slidable in a direction in which the arm 4a extends. The X-ray irradiator 3a1 and the X-ray detector 3b1 are provided face-to-face at two end portions of the holding arm 4a in the longitudinal direction. In addition, the arm supporting unit 4b holds the holding arm 4a in a slidable manner, and is turnably provided to the holding member 4c.

The holding member 4c is partially fitted into guide members (not illustrated), such as rails, which are provided to the ceiling surface in a longitudinal direction and a lateral direction of the tabletop 2a. The holing member 4c is formed to be movable by the movement mechanism 4d.

A movement manipulation detector A3 configured to detect movement manipulations by the operators such as the surgeon and the assistant is provided on a lateral side of the holding member 4c. The movement manipulation detector A3 is electrically connected to the control device 7. The movement manipulation detector A3 detects the movement manipulations by the operators, and transmits a detection signal to the control device 7. As the movement manipulation detector A3, for example, a contact manipulation switch like a touch sensor, a non-contact manipulation switch, or the like may be used. Incidentally, multiple movement manipulation detectors A3 may be provided to the holding member 4c. Alternatively, the movement manipulation detector A3 may be provided to the holding arm 4a or the arm supporting unit 4b.

The movement mechanism 4d is a mechanism configured to move the holding member 4c in the horizontal direction together with the holding arm 4a and the arm supporting unit 4b. This movement mechanism 4d is electrically connected to the control device 7, and the drive of the movement mechanism 4d is controlled by the control device 7. As the movement mechanism 4d, for example, a feed screw-type movement mechanism using a servomotor as its driving source, a linear motor-type movement mechanism using a linear motor as its driving source, or the like may be used. Incidentally, the above-described movement mechanism 4d of functions as a movement mechanism unit configured to change the positions of the X-ray irradiator 3a1 and the X-ray detector 3b1 relative to the subject P.

The image collector 5 includes: an image processing unit 5a configured to perform a process of producing X-ray images based on the X-ray doses detected by the X-ray detector 3b1; and an image storage unit 5b configured to store the produced X-ray images.

The image processing unit 5a produces the X-ray images of the subject P through various image processing steps on the basis of the X-ray doses, and transmits the produced X-ray images to the image storage unit 5b. The image storage unit 5b stores the X-ray images transmitted from the image processing unit 5a. As the image storage unit 5b, for example, a magnetic disk device, a semiconductor disk device (flash memory), and the like may be used.

The display unit 6 is a display unit configured to display various images, inclusive of the X-ray images which are transmitted from the image collector 5. As the display unit 6, for example, a liquid crystal display, a CRT (cathode ray tube) display, and the like may be used.

The control device 7 includes: a system control unit 7a configured to control the various components in the system; an X-ray control unit 7b configured to control the X-ray high-voltage generator 3a3; and a manipulation unit 7c which receives inputs from an operator such as the surgeon and the assistant.

The system control unit 7a controls the various components on the basis of various stored programs and various stored data. The system control unit 7a includes, among other things, a movement control unit 7a1 configured to control the bed 2, the image capturing device 3, the holding unit 4, and the like in accordance with the input manipulation of the operator by way of the manipulation unit 7c. The system control unit 7a further includes a region setting unit 7a2 configured to set a region of interest (ROI) in the X-ray images.

Here, the system control unit 7a outputs various instructions concerning execution of the image processing and the like to the image processing unit 5a of the image collector 5. In response to the instructions, the image processing unit 5a executes the required processing. The various components including the system control unit 7a and the image processing unit 5a may be formed from hardware including electric circuits. Alternatively, the components may be formed from software including programs for executing these functions. The components may also be formed by combining the hardware and the software.

The movement control unit 7a1 controls the movements of the various components such as the bed 2, the image capturing device 3, and the holding unit 4. The movement control unit 7a1 is capable of acquiring information on the position of the tabletop 2a on the basis of an output value from a position detection unit (for example, an encoder) which is provided to the driving source (for example, a servomotor) or the like of the bed 2. In addition, the movement control unit 7a1 is capable of acquiring information (for example, coordinates information) on the position of the X-ray detector 3b1 on the basis of an output value from a position detection unit (for example, an encoder) which is provided to the driving source (for example, a servomotor) or the like of the movement mechanism 3b2 of the X-ray detector 3b. Similarly, the movement control unit 7a1 is capable of acquiring information on the position of the holding arm 4a (for example, information on the angle of the arm, the horizontal position of the arm, and the like) on the basis of an output value from a position detection unit (for example, an encoder) which is provided to the driving source (for example, a servomotor) or the like of the movement mechanism 4d of the holding unit 4. Incidentally, the movement control unit 7a1 is further capable of recognizing the relative position between the subject P on the tabletop 2a and the X-ray detector 3b1 from the information on the position of the X-ray detector 3b1 and the information on the position of the holding arm 4a.

In addition, the movement control unit 7a1 controls an aperture value of the X-ray diaphragm 3a2. For example, when an X-ray image of a region of attention of the subject P is acquired, the aperture value of the X-ray diaphragm 3a2 is set to an appropriate aperture value for capturing the X-ray image of a predetermined region in the region of attention. Incidentally, the movement control unit 7a1 is capable of acquiring information on the aperture value of the X-ray diaphragm 3a2 on the basis of an output value from a position detection unit (for example, an encoder) which is provided to the driving source (for example, a servomotor) or the like of the X-ray diaphragm 3a2.

The X-ray control unit 7b controls various conditions of the waveform, namely the amplitude, pulse width, and the like of a voltage applied to the X-ray high-voltage generator 3a3 in order for the X-ray irradiator 3a1 to generate desired X-rays in accordance with the control by the system control unit 7a. In response to this control, the X-ray high-voltage generator 3a3 raises and rectifies the voltage, and supplies the resultant voltage to the X-ray irradiator 3a1.

The manipulation unit 7c is an input unit configured to receive input manipulations of the operators such as the surgeon and the assistant. As the manipulation unit 7c, for example, input devices such as a joystick, a keyboard, a mouse, and a footswitch can be used. The operators such as the surgeon and the assistant move the image capturing device 3, namely the X-ray irradiator 3a1 and the X-ray detector 3b1 to a desired image-capturing position by performing input manipulations of the manipulation unit 7c.

Next, descriptions will be provided for movement processes (a first movement process and a second movement process) which the X-ray diagnostic apparatus 1 performs on the X-ray detector 3b1.

### (First Movement Process)

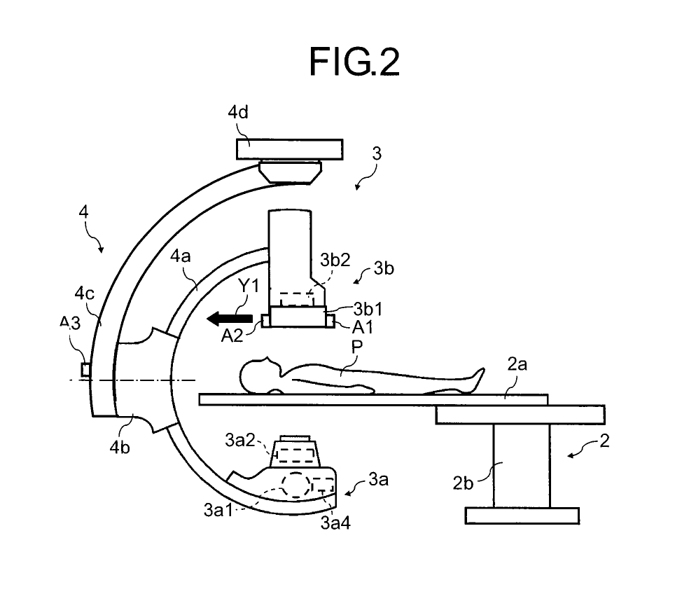

Descriptions will be provided for a movement process which is performed in a case where, as shown in FIG. 2, the surgeon moves the X-ray detector 3b1 in a direction indicated with an arrow Y1 during a hybrid surgery. The situation in this case, for example, is the situation where: the surgeon is performing a thoracotomy, and an internist is simultaneously performing a catheter treatment; the X-ray detector 3b1 hinders the surgeon from continuing the thoracotomy; and for this reason, the surgeon moves the X-ray detector 3b1 to a desired place where the detector 3b1 does not hinder the surgeon's thoracotomy.

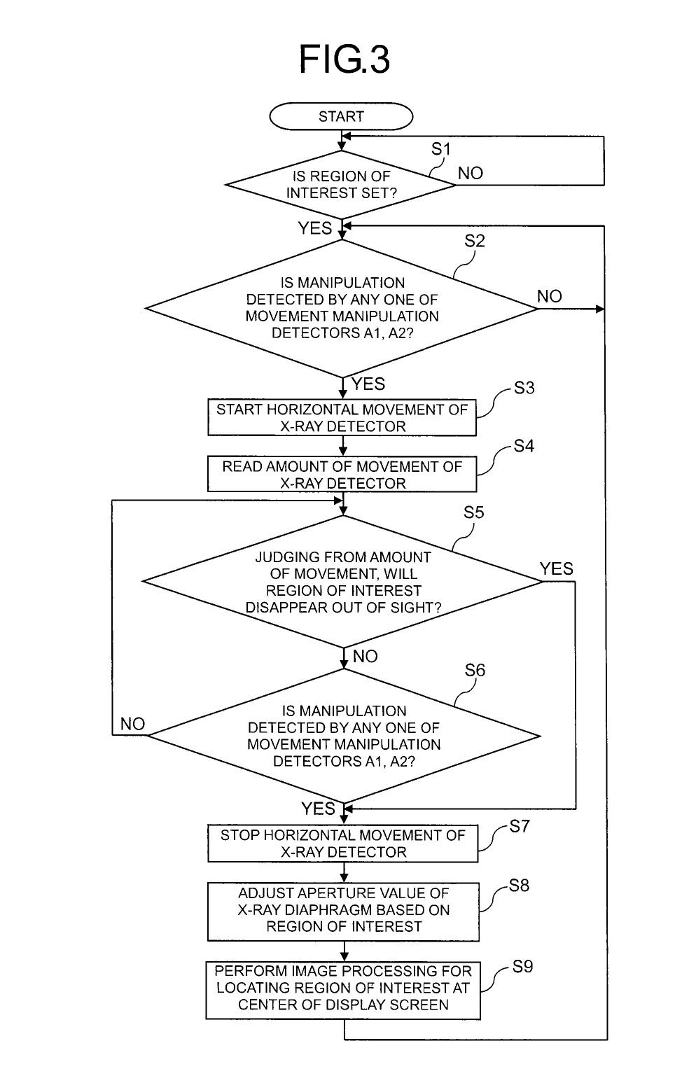

As shown in FIG. 3, it is judged whether or not a region of interest R1 is set in an X-ray image with a maximum irradiation field (step S1). This judgment is repeated until the region of interest R1 is set (if NO in step S1). Incidentally, in a case where no region of interest R1 is set, the X-ray detector 3b1 can be moved freely as usual.

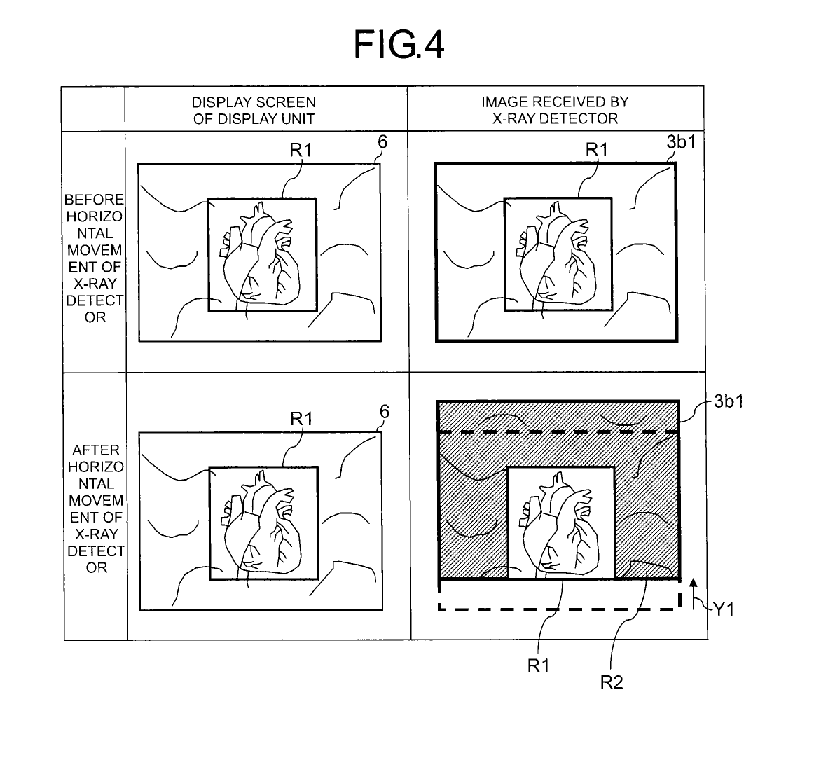

Here, let us assume that, as shown in FIG. 4 (see the upper left drawing), the region of interest R1 is set in the X-ray image displayed on the display unit 6 before the horizontal movement of the X-ray detector 3b1 takes place. To be precise, the operator such as the internist selects a desired region on the display screen of the display unit 6 by performing the input manipulation with the mouse or the like of the manipulation unit 7c. Accordingly, the selected region is set by the region setting unit 7a2 as the region of interest R1. At this time, an image received by the X-ray detector 3b1 is as shown in FIG. 4 (see the upper right drawing) (see a positional relationship between the region of interest R1 and the X-ray detector 3b1). Incidentally, although the shape of the region of interest R1 is rectangular in FIG. 4, the shape is not limited thereto. The region of interest R1 may be shaped like a circle, an ellipse or the like, for example. Otherwise, the shape of the region of interest R1 may be obtained by free-hand drawing.

Once the region of interest R1 is set as described above, a fluoroscopic image (moving image) of the region of interest R1 is obtained, and is displayed (in accordance with spot radiography, for example) on the display unit 6 while being superposed on a still image stored in advance. The internist performs the catheter treatment while visually checking the X-ray image. During this treatment, the fluoroscopy (X-ray image capturing) is repeatedly performed on the region of interest R1 alone. Only the X-ray image of the region of interest R1 is updated to a new moving image, while the X-ray image of the peripheral region is kept as the unchanged still image.

If it is judged in step S1 that the region of interest R1 is set (if YES in step S1), it is judged whether or not a movement manipulation (movement start manipulation) is detected by any one of the movement manipulation detectors A1, A2 (step S2). This judgment is repeated until the movement manipulation is detected (if NO in step S2).

Here, let us assume that the surgeon moves the X-ray detector 3b1 by manipulating any one of the movement manipulation detectors A1, A2 located on the lateral sides of the X-ray detector 3b1. Once the movement manipulation detector A1, for example, is manipulated by the operator, a detection signal is inputted from the movement manipulation detector A1 to the system control unit 7a. In response thereto, the movement control unit 7a1 of the system control unit 7a controls the movement mechanism 3ba, and thus moves the X-ray detector 3b1 in the direction indicated with the arrow Y1 (for the start of the movement).

If it is judged in step S2 that the movement manipulation is detected by any one of the movement manipulation detectors A1, A2 (if YES in step S2), the horizontal movement of the X-ray detector 3b1 is started (step S3), and the amount of movement of the X-ray detector 3b1 is read in real time (step S4). From the amount of movement, it is judged whether or not the region of interest R1 will disappear out of sight (step S5).

In this respect, if the amount of movement of the X-ray detector 3b1 becomes larger than an allowance, the region of interest R1 will go out of the display screen of the display unit 6 and will disappear out of sight. From information on the position (for example, information on the coordinates) of the region of interest R1 on the display screen, the movement control unit 7a1 can recognize how large amount of movement will cause the region of interest R1 to go out of the display screen of the display unit 6.

If, from the amount of movement of the X-ray detector 3b1, it is judged in step S5 that the region of interest R1 will not disappear out of sight (if NO in step S5), it is again judged whether or not a movement manipulation (movement stop manipulation) is detected by any one of the movement manipulation detectors A1, A2 (step S6).

In this respect, let us assume that the surgeon stops the movement of the X-ray detector 3b1 by manipulating any one of the movement manipulation detectors A1, A2 located on the lateral sides of the X-ray detector 3b1. Once the movement manipulation detector A1, for example, is manipulated again by the operator, a detection signal is inputted from the movement manipulation detector A1 to the system control unit 7a. In response thereto, the movement control unit 7a1 of the system control unit 7a controls the movement mechanism 3b2, and thus stops the movement of the X-ray detector 3b1 (for the stop of the movement).

If it is judged in step S6 that the movement manipulation is detected by any one of the movement manipulation detectors A1, A2 (if YES in step S6), the horizontal movement of the X-ray detector 3b1 is stopped (step S7). On the other hand, if it is judged that no movement manipulation is detected by either of the movement manipulation detectors A1, A2 (if NO in step S6), the process is returned to step S5.

Furthermore, if it is judged from the amount of movement of the X-ray detector 3b1 in step S5 that the region of interest R1 will disappear out of sight (if YES in step S5), the horizontal movement of the X-ray detector 3b1 is stopped at the current position (step S7).

In other words, if it is judged from the amount of movement of the X-ray detector 3b1 that the region of interest R1 will not disappear out of sight (if NO in step S5), the X-ray detector 3b1 horizontally moves to the desired position. On the other hand, if it is judged from the amount of movement of the X-ray detector 3b1 that the region of interest R1 will disappear out of sight (if YES in step S5), the X-ray detector 3b1 moves to a position which makes the amount of movement to the maximum while keeping the entirety of the region of interest R1 within sight instead of moving to the desired position (for the limitation of the movement).

In this respect, as shown in FIG. 4 (see the lower right drawing), the X-ray detector 3b1 is moved by the movement mechanism 3b2 in the direction indicated with the arrow Y1, to a position in which the entire region of interest R1 is prevented from disappearing out of sight. During this movement, even the slightest part of the region of interest R1 does not disappear from view of the internist. In other words, while keeping the region of interest R1 visible, the X-ray detector 3b1 moves to the position in which the X-ray detector 3b1 keeps the entirety of the region of interest R1 within sight after the stop.

In some cases, the X-ray irradiator 3a1 and the X-ray diaphragm 3a2 may be horizontally moved by the movement mechanism 3a4 in accordance with the above-mentioned horizontal movement of the X-ray detector 3b1. For example, if the relative position between the X-ray detector 3b1 and the X-ray irradiator 3a1 at the start of image capturing (in the image-capturing starting state) needs to be retained, then the X-ray irradiator 3a1 and the X-ray diaphragm 3a2 are horizontally moved, in accordance with the horizontal movement of the X-ray detector 3b1, in such a way as to retain the relative position between the X-ray detector 3b1 and the X-ray irradiator 3a1 in the image-capturing starting state.

After the above-described step S7, the aperture value of the X-ray diaphragm 3a2 is adjusted on the basis of the region of interest R1 (step S8); an image process is performed for locating the region of interest R1 almost at the center of the display screen (step S9); and the movement process is returned to step S2. If another movement manipulation is detected anew by any one of the movement manipulation detector A1, A2 (if YES in step S2), the above-described process is repeated.

In step S8, the X-ray diaphragm 3a2 is controlled in such a way as to irradiate only the region of interest R1 with the X-rays, and the aperture value of the X-ray diaphragm 3a2 is accordingly adjusted. Thus, as shown in FIG. 4 (see the lower right drawing), a blocking region R2 (see a hatched region in the lower right drawing) for blocking the X-rays is formed. As a result, unnecessary radioactive exposure is inhibited, and a reduction in radioactive exposure can be achieved. Incidentally, if the region of interest R1 in this state (see the lower right drawing) is displayed as it is on the display unit 6, it is difficult for the internist to recognize the region of interest R1 since the region of interest R1 is situated close to the lower end of the display screen of the display unit 6.

With this taken into consideration, in step S9, the X-ray image is processed by the image processing unit 5a in order that the region of interest R1 should be placed almost at the center of the display screen of the display unit 6. Thus, as shown in FIG. 4 (see the lower left drawing), the region of interest R1 is situated almost at the center of the display screen even after the horizontal movement of the X-ray detector 3b1. This makes it possible for the internist to visually recognize the region of interest R1 in the display screen with ease, and to perform the catheter treatment efficiently.

Here, if the operators such as the surgeon and the assistant instruct cancellation of the movement of the X-ray detector 3b1 by performing an input manipulation with the manipulation unit 7c, the X-ray detector 3b1 moves to its original position. In this case, information on the position of the X-ray detector 3b1 before its movement is stored in the movement control unit 7a1. The information on the position is retrieved in accordance with the input manipulation with the manipulation unit 7c, and the X-ray detector 3b1 is moved by the movement mechanism 3b2 to its original position on the basis of the thus-retrieved information on the position.

### (Second Movement Process)

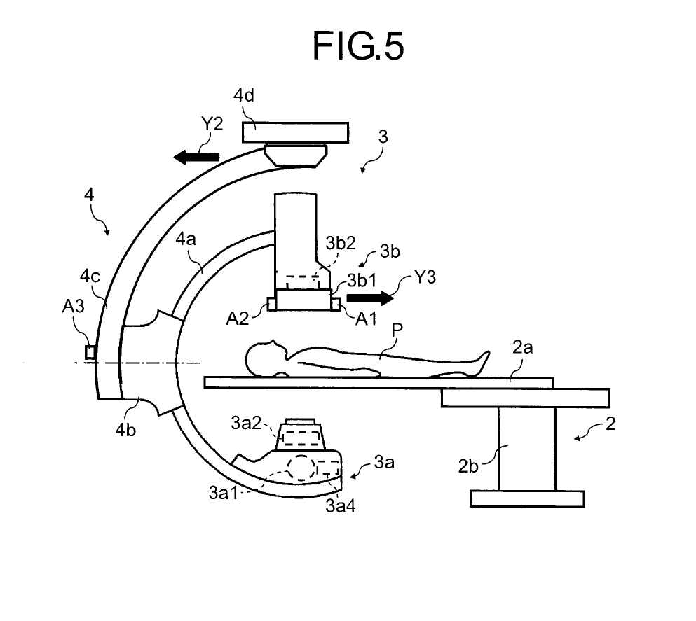

Descriptions will be provided for another movement process which is performed on the X-ray detector 3b1 (a movement in a direction indicated with an arrow Y3) in a case where, as shown in FIG. 5, the surgeon moves the holding unit 4 in a direction indicated with an arrow Y2 during a hybrid surgery. The situation in this case, for example, is the situation where: the surgeon is performing a thoracotomy as in the case previously described, and the internist is simultaneously performing a catheter treatment; the holding unit 4 (for example, the holding arm 4a) hinders the surgeon from continuing the thoracotomy; and for this reason, the surgeon moves the holding unit 4 to a desired place where the holding unit 4 does not hinder the surgeon's thoracotomy.

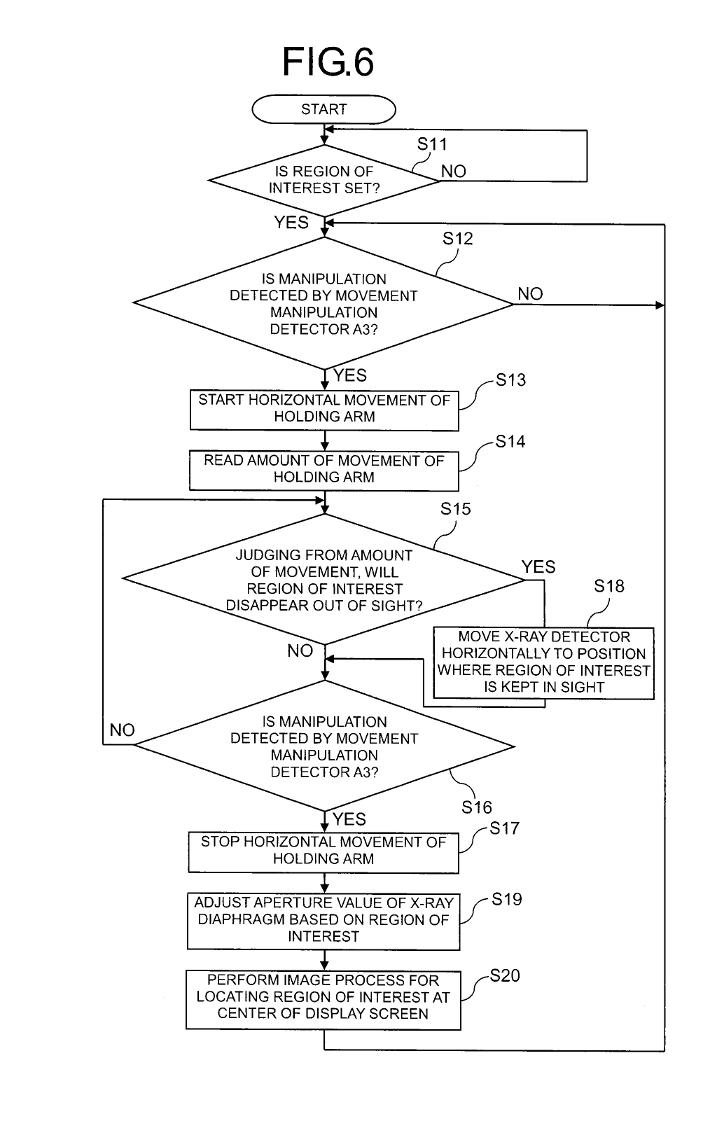

As shown in FIG. 6, it is judged whether or not a region of interest R1 is set in an X-ray image representing the entirety of the X-ray irradiation field (step S11). This judgment is repeated until the region of interest R1 is set (if NO in step S11). Incidentally, in a case where no region of interest R1 is set, the holding arm 4a can be moved freely as usual.

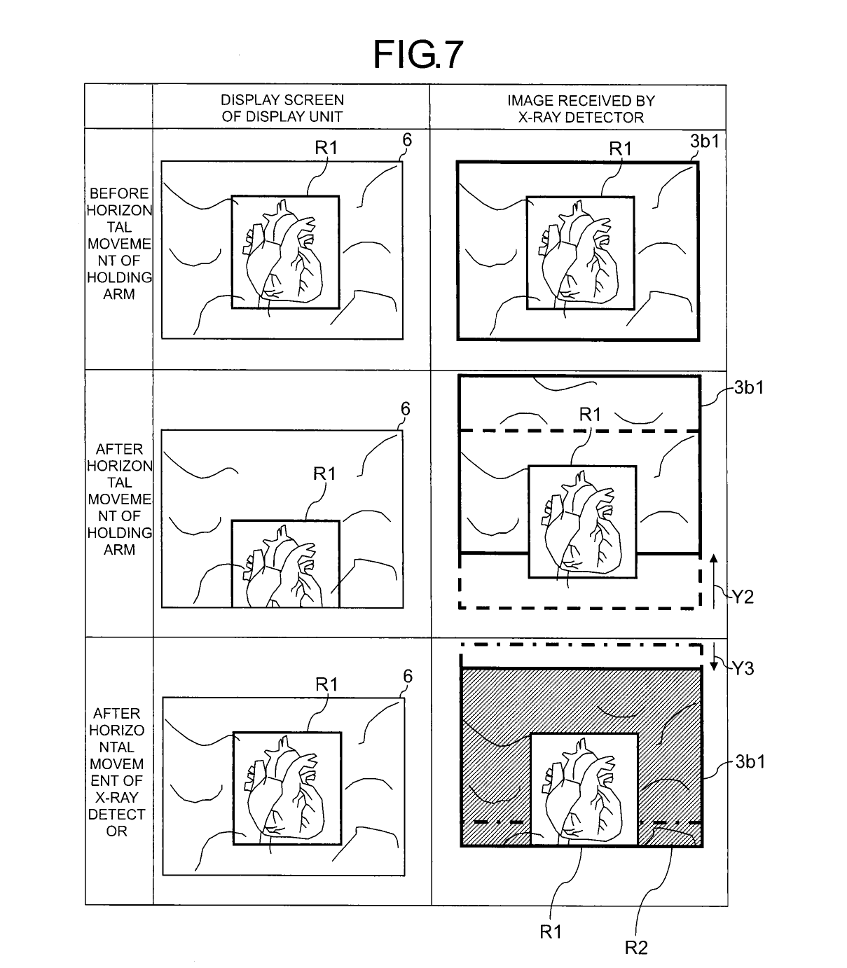

Here, let us assume that, as shown in FIG. 7 (see the upper left drawing), the region of interest R1 is set in the X-ray image displayed on the display unit 6 before the horizontal movement of the holding arm 4a. To be precise, the operator such as the internist selects a desired region on the display screen of the display unit 6 by performing the input operation with the mouse or the like of the manipulation unit 7c. Accordingly, the selected region is set by the region setting unit 7a2 as the region of interest R1. At this time, an image received by the X-ray detector 3b1 is as shown in FIG. 7 (see the upper right drawing) (see a positional relationship between the region of interest R1 and the X-ray detector 3b1). Incidentally, although the shape of the region of interest R1 is rectangular in FIG. 7, the shape is not limited thereto. The region of interest R1 may be shaped like a circle, an ellipse or the like, for example. Otherwise, the shape of the region of interest R1 may be obtained by free-hand drawing.

Once the region of interest R1 is set as described above, a fluoroscopic image (moving image) of the region of interest R1 is obtained, and is displayed (in accordance with spot radiography, for example) on the display unit 6 while being superposed on a still image stored in advance, as in the case previously described. The internist performs the catheter treatment while visually checking the X-ray image. During this treatment, the fluoroscopy (X-ray image capturing) is repeatedly performed on the region of interest R1 alone. Only the X-ray image of the region of interest R1 is updated to a new moving image, while the X-ray image of the peripheral region is kept at the unchanged still image.

If it is judged in step S11 that the region of interest R1 is set (if YES in step S11), it is judged whether or not a movement manipulation (movement start manipulation) is detected by the movement manipulation detector A3 (step S12). This judgment is repeated until the movement manipulation is detected (if NO in step S12).

Here, let us assume that the surgeon moves the holding unit 4, particularly the holding arm 4a, by manipulating the movement manipulation detector A3 located on the lateral side of the holding member 4c of the holding unit 4. Once the movement manipulation detector A3, for example, is manipulated by the operator, a detection signal is inputted from the movement manipulation detector A3 to the system control unit 7a. In response thereto, the movement control unit 7a1 of the system control unit 7a controls the movement mechanism 4d, and thus moves the holding unit 4 in the direction indicated with the arrow Y2 (for the start of the movement).

If it is judged in step S12 that the movement manipulation is detected by the movement manipulation detector A3 (if YES in step S12), the horizontal movement of the holding unit 4 is started (step S13), and the amount of movement of the holding unit 4, namely the holding arm 4a, is read in real time (step S14). From the amount of movement, it is judged whether or not the region of interest R1 will disappear out of sight (step S15).

In this respect, if the amount of movement of the holding arm 4a, namely the X-ray detector 3b1, becomes larger than an allowance, the region of interest R1 will go out of the display screen of the display unit 6 and will disappear out of sight. From information on the position (for example, information on the coordinates) of the region of interest R1 on the display screen, the movement control unit 7a1 can recognize how large amount of movement will cause the region of interest R1 to go out of the display screen of the display unit 6.

If, from the amount of movement of the holding arm 4a, it is judged in step S15 that the region of interest R1 will not disappear out of sight (if NO in step S15), it is again judged whether or not a movement manipulation (movement stop manipulation) is detected by the movement manipulation detector A3 (step S16).

In this respect, let us assume that the surgeon stops the movement of the holding unit 4, particularly the holding arm 4a, by manipulating the movement manipulation detector A3 located on the lateral side of the supporting member 4c of the holding unit 4. Once the movement manipulation detector A3, for example, is manipulated again by the operator, a detection signal is inputted from the movement manipulation detector A3 to the system control unit 7a. In response thereto, the movement control unit 7a1 of the system control unit 7a controls the movement mechanism 4d, and thus stops the movement of the holding unit 4 (for the stop of the movement).

If it is judged in step S16 that the movement manipulation is detected by the movement manipulation detector A3 (if YES in step S16), the horizontal movement of the holding arm 4a is stopped (step S17). On the other hand, if it is judged that no movement manipulation is detected by the movement manipulation detector A3 (if NO in step S16), the process is returned to step S15.

Furthermore, if it is judged from the amount of movement of the holding arm 4a in step S15 that the region of interest R1 will disappear out of sight (if YES in step S15), the X-ray detector 3b1 horizontally moves to a position where the region of interest R1 is kept within sight (in step S18), and the process proceeds to step S16.

In other words, if it is judged from the amount of movement of the holding arm 4a that the region of interest R1 will not disappear out of sight (if NO in step S15) as described above, the holding arm 4a horizontally moves to the desired position. On the other hand, if it is judged from the amount of movement of the holding arm 4a that the region of interest R1 will disappear out of sight (if YES in step S15), the X-ray detector 3b1 horizontally moves to the position where the entirety of the region of interest R1 is kept within sight. Incidentally, the holding arm 4a is horizontally moved within a range in which the horizontal movement of the X-ray detector 3b1 keeps the region of interest R1 within sight.

In this respect, as shown in FIG. 7 (see the center right drawing), in accordance with the movement of the holding arm 4a by the movement mechanism 4d, the X-ray detector 3b1 moves in the direction indicated with the arrow Y2 to the position where the movement of the X-ray detector 3b1 makes part of the region of interest R1 out of sight. During this movement, as shown in FIG. 7 (see the center left drawing), a state where the part of the region of interest R1 disappear out of sight occurs in the display screen of the display unit 6. As shown in FIG. 7 (see the lower right drawing), however, the X-ray detector 3b1 is moved by the movement mechanism 3b2 in the direction indicated with the arrow Y3 to the position where the X-ray detector 3b1 keeps the entirety of the region of interest R1 within sight. Thus, the entirety of the region of interest R1 is displayed on the display unit 6. For this reason, the internist can visually recognize the entirety of the region of interest R1.

In some cases, the X-ray irradiator 3a1 and the X-ray diaphragm 3a2 may be horizontally moved by the movement mechanism 3a4 in accordance with the above-mentioned horizontal movement of the X-ray detector 3b1. For example, if the relative position between the X-ray detector 3b1 and the X-ray irradiator 3a1 at the start of image capturing (in the image-capturing starting state) needs to be retained, then the X-ray irradiator 3a1 and the X-ray diaphragm 3a2 are horizontally moved, in accordance with the horizontal movement of the X-ray detector 3b1, in such a way as to retain the relative position between the X-ray detector 3b1 and the X-ray irradiator 3a1 in the image-capturing starting state.

After the above-described step S17, the aperture value of the X-ray diaphragm 3a2 is adjusted on the basis of the region of interest R1 (step S19); an image process is performed for locating the region of interest R1 almost at the center of the display screen (step S9); and the movement process is returned to step S12. If another movement manipulation is detected anew by the movement manipulation detector A3 (if YES in step S12), the above-described process is repeated.

As in the above-described step S8, in step S19, the X-ray diaphragm 3a2 is controlled in such a way as to irradiate only the region of interest R1 with the X-rays, and the aperture value of the X-ray diaphragm 3a2 is accordingly adjusted. Thus, as shown in FIG. 7 (see the lower right drawing), a blocking region R2 (see a hatched region in the lower right drawing) for blocking the X-rays is formed. As a result, unnecessary radioactive exposure is inhibited, and a reduction in radioactive exposure can be achieved. Incidentally, if the region of interest R1 in this state (see the lower right drawing) is displayed as it is on the display unit 6, it is difficult for the internist to recognize the region of interest R1 since the region of interest R1 is situated close to the lower end of the display screen of the display unit 6.

With this taken into consideration, like in step S9 described above, the X-ray image is processed by the image processing unit 5a in step S20 in order that the region of interest R1 should be placed almost at the center of the display screen of the display unit 6. Thus, as shown in FIG. 7 (see the lower left drawing), the region of interest R1 is situated almost at the center of the display screen even after the horizontal movement of the X-ray detector 3b1. This makes it possible for the internist to visually recognize the region of interest R1 in the display screen with ease, and to perform the catheter treatment efficiently.

Here, if the operators such as the surgeon and the assistant instruct cancellation of the movement of the X-ray detector 3b1 by performing an input manipulation with the manipulation unit 7c, the X-ray detector 3b1 moves to its original position. In this case, information on the positions of the X-ray detector 3b1 and the holding arm 4a before their movements or the like is stored in the movement control unit 7a1. The information on the positions is retrieved in accordance with the input manipulation with the manipulation unit 7c, and the X-ray detector 3b1 is moved by the movement mechanism 3b2 to its original position on the basis of the thus-retrieved information on the positions. Further, the holding unit 4 is moved by the movement mechanism 4d.

In the foregoing embodiment, as described above, the region of interest R1 is set in the X-ray image displayed in the display screen of the display unit 6; and the movement mechanism unit (including the movement mechanism 3b2 and the movement mechanism 4d, for example) are controlled in such a way as to keep the region of interest R1 in the display screen. For this reason, even if the surgeon moves the X-ray detector 3b1 at his or her will during the operation because the X-ray detector 3b1 hinders the surgeon's operation, the internist can visually recognize the region of interest R1. As a result, the surgeon can move the X-ray detector 3b1 as needed, while the internist can visually recognize the region of interest R1 securely. This makes it possible for the surgeon and the internist to efficiently perform their work at the same time.

In addition, the movement mechanism unit is formed from the movement mechanism (first movement mechanism) 3b2 and the movement mechanism (second movement mechanism) 4d. When the X-ray detector 3b1 moves, either or both of the movement mechanism 3b2 and the movement mechanism 4d are controlled in accordance with the movement of the X-ray detector 3b1 in such a way as to keep the region of interest R1 in the display screen. This makes it possible to easily keep the region of interest R1 in the display screen.

Furthermore, when the X-ray detector 3b1 moves, the X-ray image is processed in such a way that the region of interest R1 is placed almost at the center of the display screen. This makes it possible for the internist to visually recognize the region of interest R1 in the display screen with ease, and to efficiently perform the work such as the catheter treatment.

Moreover, when the movement manipulation for instructing the movement of the X-ray detector 3b1 is detected by any one of the movement manipulation detectors A1, A2, the movement of the X-ray detector 3b1 by the movement mechanism 3b2 is controlled. This makes it possible for the surgeon to move the X-ray detector 3b1 by only manipulating one of the movement manipulation detectors A1, A2, A3. Accordingly, the work efficiency of the surgeon can be improved.

What is more, the X-ray diaphragm 3a2 is controlled in such a way that the irradiation of the X-rays is targeted at the region of interest R1. For this reason, the unnecessary radioactive exposure can be inhibited and a reduction in radioactive exposure can be realized.

Meanwhile, when the manipulation unit 7c receives a movement cancellation manipulation from the manipulation unit 7c which instructs the cancellation of the movement of the X-ray detector 3b1, the movement mechanism 3b2 is controlled in such a way as to move the X-ray detector 3b1 to its original position. For this reason, when the X-ray detector 3b1 does not hinder the work of the operators such as the surgeon and the assistant any more, the operators can easily return the X-ray detector 3b1 to its original position. Accordingly, the work efficiency of the surgeon can be improved.

In addition, the movement mechanism unit is formed from the movement mechanism (first movement mechanism) 3b2 and the movement mechanism (third movement mechanism) 3a4. The movement mechanism 3b2 and the movement mechanism 3a4 are controlled in such a way that the X-ray irradiator 3a1 moves in accordance with the movement of the X-ray detector 3b1. This makes it possible to retain, for example, the relative position between the X-ray detector 3b1 and the X-ray irradiator 3a1 at the start of image capturing (in the image-capturing starting state).

In the embodiment, only the X-ray detector 3b1 is moved during the hybrid surgery without moving the tabletop 2a of the bed 2. However, the present invention is not limited to the foregoing. For example, even in a case where the tabletop 2a moves, a process like the above-described process can be executed by using an amount of movement of the tabletop 2a. Nonetheless, it is rare to move the tabletop 2a during a surgical operation such as a hybrid operation.

In addition, although the foregoing descriptions have been provided for the embodiment in which the X-ray irradiator 3a1 is placed under the tabletop 2a while the X-ray detector 3b1 is placed over the tabletop 2a, the present invention is not limited to this configuration. For example, the X-ray irradiator 3a1 and the X-ray detector 3b1 may be arranged the other way around. Their positions may be changed freely as long as their positional relationship remains to face each other. When their positions are changed, the X-ray irradiator 3a1, instead of the X-ray detector 3b1, may hinder the surgeon's work. Even in this case, the X-ray irradiator 3a1 can be moved like the X-ray detector 3b1 of the foregoing embodiment. Hence, it is possible to obtain the same effects as those of the foregoing embodiment.

Furthermore, although the C-arm is used in the embodiment as an example of the holding arm 4a, the present invention is not limited to this configuration. For example, two arms including a robot arm configured to hold the X-ray irradiator 3a1 and a robot arm configured to hold the X-ray detector 3b1 may be used (note that these two arms move independently of each other). Likewise, various types of arms are applicable to the invention.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray diagnostic apparatus comprising:
an X-ray irradiator configured to irradiate a subject with X-rays;
an X-ray detector configured to detect the X-rays emitted from the X-ray irradiator and passing through the subject;
a movement mechanism unit configured to change a position of any one of the X-ray detector and the X-ray irradiator relative to the subject;
a display unit configured to display an X-ray image based on the X-rays detected by the X-ray detector;
a region setting unit configured to set a region of interest in the X-ray image displayed in a display screen of the display unit; and
a movement control unit configured to control the movement mechanism unit in such a way that the region of interest set by the region setting unit is kept in the display screen.

2. The X-ray diagnostic apparatus of claim 1, wherein
the movement mechanism unit comprises:
a first movement mechanism configured to move the X-ray detector in a plane; and
a second movement mechanism configured to move a holding arm that holds the X-ray irradiator and the X-ray detector, and
the movement mechanism unit controls at least one of the first movement mechanism and the second movement mechanism in accordance with the movement of the X-ray detector in such a way that the region of interest is kept in the display screen.

3. The X-ray diagnostic apparatus of claim 1, further comprising an image processing unit configured to process the X-ray image when the X-ray detector moves, in such a way that the region of interest is located almost at the center of the display screen.

4. The X-ray diagnostic apparatus of claim 2, further comprising an image processing unit configured to process the X-ray image when the X-ray detector moves, in such a way that the region of interest is located almost at the center of the display screen.

5. The X-ray diagnostic apparatus of claim 1, further comprising a movement manipulation detector configured to detect a movement manipulation for instructing a movement of the X-ray detector, wherein
the movement control unit controls the movement mechanism unit if the movement manipulation is detected by the movement manipulation detector.

6. The X-ray diagnostic apparatus of claim 2, further comprising a movement manipulation detector configured to detect a movement manipulation for instructing a movement of the X-ray detector, wherein
the movement control unit controls the movement mechanism unit if the movement manipulation is detected by the movement manipulation detector.

7. The X-ray diagnostic apparatus of claim 1, further comprising an X-ray diaphragm configured to adjust a field of irradiation of the X-rays emitted from the X-ray irradiator, wherein
the movement control unit controls the X-ray diaphragm in such a way that the irradiation of the X-rays is targeted at the region of interest.

8. The X-ray diagnostic apparatus of claim 2, further comprising an X-ray diaphragm configured to adjust a field of irradiation of the X-rays emitted from the X-ray irradiator, wherein
the movement control unit controls the X-ray diaphragm in such a way that the irradiation of the X-rays is targeted at the region of interest.

9. The X-ray diagnostic apparatus of claim 1, further comprising a manipulation unit configured to receive a cancellation manipulation for instructing cancellation of a movement of the X-ray detector,
wherein
if the cancellation manipulation is done by use of the manipulation unit, the movement control unit controls the movement mechanism unit in order to move the X-ray detector to its original position.

10. The X-ray diagnostic apparatus of claim 2, further comprising a manipulation unit configured to receive a cancellation manipulation for instructing cancellation of a movement of the X-ray detector,
wherein
if the cancellation manipulation is done by use of the manipulation unit, the movement control unit controls the movement mechanism unit in order to move the X-ray detector to its original position.

11. The X-ray diagnostic apparatus of claim 1, wherein
the movement mechanism unit comprises:
a first movement mechanism configured to move the X-ray detector in a plane; and
a third movement mechanism configured to move the X-ray irradiator in a plane, and
the movement control unit controls the first movement mechanism and the third movement mechanism in such a way that the X-ray irradiator moves in accordance with the movement of the X-ray detector.
